(19) United States
(12) Patent Application Publication
(10) Pub. No.: US 2022/0193299 A1
(43) Pub. Date: Jun. 23, 2022

(54) Air Sterilizing And Lighting Apparatus

(71) Applicant: Jihun LEE, Daegu (KR)

(72) Inventor: Jihun LEE, Daegu (KR)

(21) Appl. No.: 17/152,528

(22) Filed: Jan. 19, 2021

(30) Foreign Application Priority Data
Dec. 23, 2020 (KR) 10-2020-0181675

(51) Int. Cl. A61L 9/20

(52) U.S. Cl. CPC A61L 9/205; A61L 2209/12

(57) ABSTRACT

Disclosed is an air sterilizing and lighting apparatus including a main housing, a subsidiary housing, a substrate and a cover. The air sterilizing and lighting apparatus is configured such that LEDs provided on the lower surface of the substrate emit light, and indoor air introduced into the main housing due to the air blast action of an air blower fan fixedly coupled to a coupling groove of the main housing is sterilized due to the photocatalytic reaction of titanium oxide plates and due to radiation of ultraviolet rays from UV LEDs directed to various regions of the sterilization paths while passing through the sterilization paths formed in a zigzag form by heat-dissipation and guide plates provided at both sides of the coupling groove, and is then discharged to an indoor space through exits, thereby being capable of more rapidly sterilizing the indoor air over a wide area.

### BACKGROUND OF THE INVENTION

### Field of the Invention

The present invention relates to an air sterilizing and lighting apparatus which may serve both to provide lighting and to sterilize indoor air.

### Description of the Related Art

Recently, as the average global temperature is rising due to global warming and the amount of fine dust is increasing, not only residential spaces in general homes but also the interiors of various transportation means, such as subways, ships, and aircrafts, are being changed to environments appropriate to breeding of bacteria and thus require sterilization, and the interiors of various household appliances, such as refrigerators, dish washers, etc., in which bacteria proliferate, also require sterilization.

Therefore, air sterilizing and lighting apparatuses which may serve both to provide lighting and to sterilize indoor air were disclosed in Korean Patent Registration No. 10-1066067 and Korean Patent Registration No. 10-1241660.

However, the above air sterilizing and lighting apparatuses are configured such that UV LEDs are simply installed at positions avoiding a lamp unit for lighting without any unit for the flow of indoor air and thus are not capable of effectively sterilizing ambient air using only ultraviolet rays emitted from the UV LEDs, and sterilizes only a local area in an indoor space, in which air flow is stagnant, and thus are not capable of expecting sterilization of a wide area and have problems, such as sterilization efficiency degradation.

### RELATED ART DOCUMENT

### Patent Document

(Patent Document 1) Korean Patent Registration No. 10-1066067 (Published Date: Sep. 20, 2011)

(Patent Document 2) Korean Patent Registration No. 10-1241660 (Published Date: Mar. 11, 2013)

### SUMMARY OF THE INVENTION

Therefore, the present invention has been made in view of the above problems, and it is an object of the present invention to provide an air sterilizing and lighting apparatus which may more rapidly sterilize indoor air over a wide area in addition to providing a lighting function.

In accordance with the present invention, the above and other objects can be accomplished by the provision of an air sterilizing and lighting apparatus including a main housing, a subsidiary housing, a substrate and a cover, and is configured such that LEDs provided on the lower surface of the substrate emit light so as to exhibit a lighting function, and indoor air introduced into the main housing due to the air blast action of an air blower fan fixedly coupled to a coupling groove of the main housing is sterilized due to the photocatalytic reaction of titanium oxide plates installed at the sterilization paths and due to radiation of ultraviolet rays from UV LEDs directed to various regions of the sterilization paths while passing through the sterilization paths formed in a zigzag form by heat-dissipation and guide plates provided at both sides of the coupling groove, and is then discharged to an indoor space through exits.

### BRIEF DESCRIPTION OF THE DRAWINGS

The above and other objects, features and other advantages of the present invention will be more clearly understood from the following detailed description taken in conjunction with the accompanying drawings, in which:

FIG. 1 is a bottom perspective view of an air sterilizing and lighting apparatus according to the present invention;

FIG. 2 is a partially exploded bottom perspective view of the air sterilizing and lighting apparatus according to the present invention;

FIG. 3 is a completely exploded bottom perspective view of the air sterilizing and lighting apparatus according to the present invention;

FIG. 4 is a perspective view of a substrate used in the air sterilizing and lighting apparatus according to the present invention; and

FIG. 5 is a cross-sectional view of the air sterilizing and lighting apparatus according to the present invention.

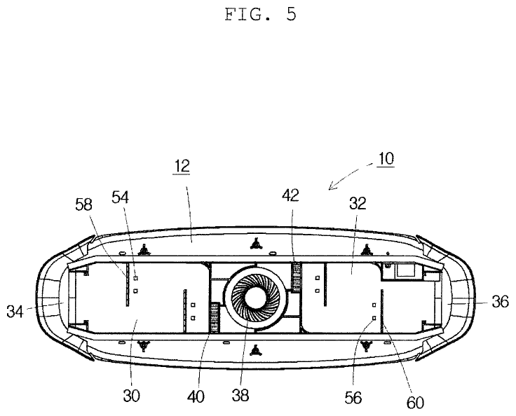

### DETAILED DESCRIPTION OF THE INVENTION

Hereinafter reference will now be made in detail to various embodiments of the present invention, examples of which are illustrated in the accompanying drawings and described below. While the invention will be described in conjunction with exemplary embodiments, it will be understood that the present description is not intended to limit the invention to the exemplary embodiments.

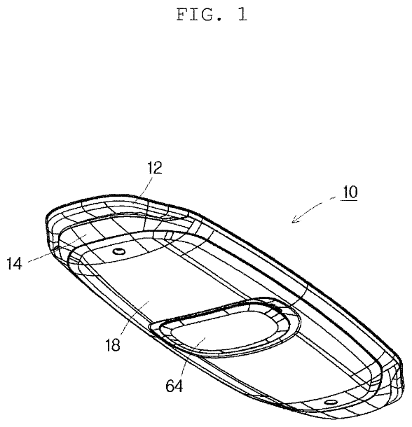

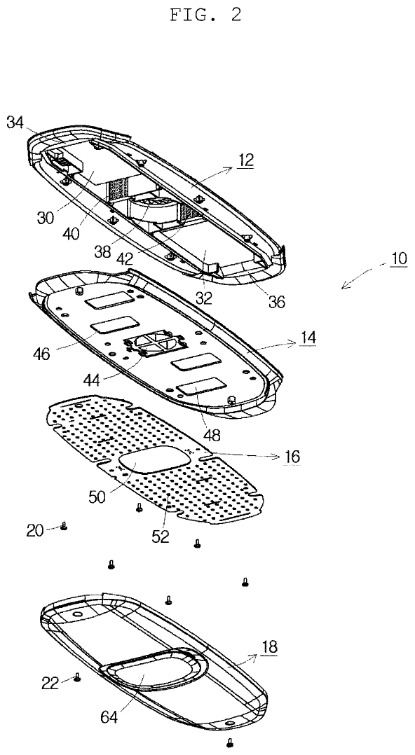

FIG. 1 is a bottom perspective view of an air sterilizing and lighting apparatus according to the present invention, and FIG. 2 is a partially exploded bottom perspective view of the air sterilizing and lighting apparatus according to the present invention. An air sterilizing and lighting apparatus 10 according to the present invention is configured such that a main housing 12 extending laterally, a subsidiary housing 14 extending laterally and provided under the main housing 12, a substrate 16, and a cover 18 are sequentially coupled so as to be fixedly connected by bolts 20 and 22.

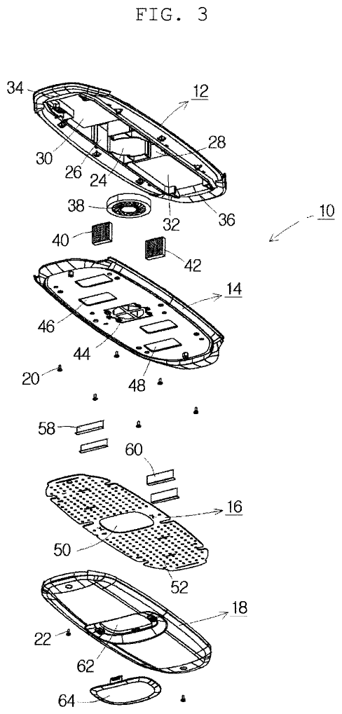

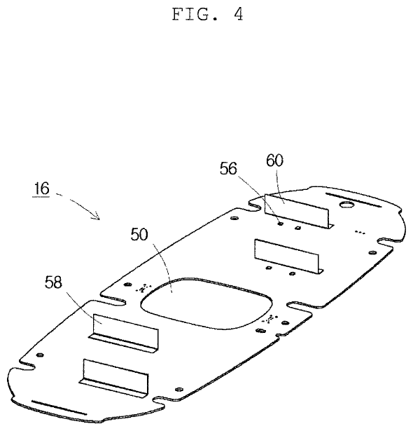

FIG. 3 is a completely exploded bottom perspective view of the air sterilizing and lighting apparatus according to the present invention, and FIG. 4 is a perspective view of a substrate used in the air sterilizing and lighting apparatus according to the present invention. The main housing 12 is configured such that a coupling groove 24 is formed in the center of the bottom portion of the main housing 12, sterilization paths 30 and 32 extending from entrances 26 and 28, which are narrowed by respective diaphragms, are provided at both sides of the coupling groove 24, exits 34 and 36 are provided at the ends of the sterilization paths 30 and 32 opposite the entrances 26 and 28, and an air blower fan 38 is fixedly coupled into the coupling groove 24, and titanium oxide plates 40 and 42 having vent holes arranged densely are fixedly coupled to the entrances 26 and 28 of the sterilization paths 30 and 32 provided at both ends of the coupling groove 24, so that indoor air introduced into the main housing 12 by the air blower fan 38 is sterilized while passing through the vent holes of the titanium oxide plates 40 and 42 and the sterilization paths 30 and 32, and is then discharged to an indoor space through the exits 34 and 36.

However, the titanium oxide plates 40 and 42 are not necessarily installed at the entrances 26 and 28 of the sterilization paths 30 and 32 of the main housing 12, and may be installed inside the sterilization paths 30 and 32 or installed at the exits 34 and 36 of the sterilization paths 30 and 32.

The subsidiary housing 14 is configured such that an inlet 44 for the flow of indoor air is formed at the center of the subsidiary housing 14 and a plurality of through holes 46 and 48 for exposure of the sterilization paths 30 and 32 of the main housing 12 are formed at designated positions of both sides of the inlet 44, and the subsidiary housing 14 is fixedly coupled to the bottom portion of the main housing 12.

The substrate 16 is configured such that an inlet 50 for in the flow of indoor air is formed at the center of the substrate 16, a plurality of LEDs 52 is fixedly installed on the lower surface of the substrate 16, a plurality of UV LEDs 54 and 56 and heat-dissipation and guide plates 58 and 60 are fixedly installed at designated positions of both sides of the upper surface of the substrate 16, and the substrate 16 is fixedly connected to the bottom portion of the main housing 12 across the subsidiary housing 14 using the bolts 20.

Here, the inlet 50 formed at the center of the substrate 16 communicates with the inlet 44 of the subsidiary housing 14, the UV LEDs 54 and 56 are directed to various regions of the sterilization paths 30 and 32 of the main housing 12 via the through holes 46 and 48 formed through the left and right sides of the subsidiary housing 14, and the heat-dissipation and guide plates 58 and 60 enter the insides of the sterilization paths 30 and 32 via the through holes 46 and 48 of the subsidiary housing 14 and thus allow the sterilization paths 30 and 32 to be formed in a zigzag form.

Because the UV LEDs 54 and 56 are installed on the substrate 16 for the purpose of ease of manufacture and the heat-dissipation and guide plates 58 and 60 are installed on the substrate 16 so as to also exhibit a heat dissipation function, the UV LEDs 54 and 56 and the heat-dissipation and guide plates 58 and 60 may be installed on the subsidiary housing 14 or the main housing 12 rather than the substrate 16.

The cover 18 is configured such that an inlet 62 for the flow of indoor air is formed at the center of the cover 18 so as to communicate with the inlet 50 of the substrate 16, and the cover 18 is fixedly connected to the bottom portion of the substrate 16 using the bolts 22.

Further, a subsidiary cover 64 may be coupled to the inlet 62 of the cover 18 so as to remain a designated distance from the cover 18 using hanging holes and hanging pieces, thereby exposing the inlet 62 in the lateral direction.

When the air sterilizing and lighting apparatus according to the present invention is installed not only in residential spaces in general homes but also in the interiors of various transportation means, such as subways, ships, and aircrafts, or the interiors of various household appliances, such as refrigerators, dish washers, etc., and then power is applied thereto, the LEDs 52 installed on the lower surface of the substrate 16 emit light and thus exhibit the lighting function.

Here, heat generated from the LEDs 52 when the LEDs 52 emit light is rapidly dissipated by the heat-dissipation and guide plates 58 and 60 fixedly installed at both sides of the upper surface of the substrate 16 so as to be located inside the sterilization paths 30 and 32 of the main housing 12 via the through holes 46 and 48 of the subsidiary housing 14, and thus, the service lives of the LEDs 52 and the UV LEDs 54 and 56 may be prolonged without using separate units for dissipating heat or securing an additional space.

Particularly, when the air blower fan 38 fixedly coupled to the center of the main housing 12 is operated, indoor air is introduced into the main housing 12 through the inlets 44, 50 and 62 of the subsidiary housing 14, the substrate 16 and the cover 18, and is primarily sterilized due to the photocatalytic reaction of the titanium oxide plates 40 and 42 while passing through the vent holes densely formed through the titanium oxide plates 40 and 42 fixedly coupled to the entrances 26 and 28 of the sterilization paths 30 and 32, the indoor air primarily sterilized by the titanium oxide plates 40 and 42 is secondarily sterilized due to radiation of ultraviolet rays from the UV LEDs 54 and 56 directed to various regions of the sterilization paths 30 and 32 while passing through the sterilization paths 30 and 32 formed in a zigzag form by the heat-dissipation and guide plates 58 and 60, and the sterilized indoor air is discharged to the indoor space through the exits 34 and 36 provided at the ends of the sterilization paths 30 and 32, thereby allowing the indoor air to be more rapidly sterilized over a wide area.

As is apparent from the above description, an air sterilizing and lighting apparatus according to the present invention is configured such that indoor air introduced into a main housing due to the air blast action of an air blower fan is sterilized due to the photocatalytic reaction of titanium oxide plates installed at sterilization paths and due to radiation of ultraviolet rays from UV LEDs directed to various regions of the sterilization paths while passing through the sterilization paths provided at both sides of the air blower fan in a zigzag form, thereby being capable of more rapidly sterilizing the indoor air over a wide area.

Further, a plurality of heat-dissipation and guide plates configured to form the sterilization paths in a zigzag form is fixedly attached to a substrate so as to rapidly dissipate heat generated from LEDs when the LEDs emit light, thereby being capable of ensuring the service lives of the LEDs and the UV LEDs without using separate units for dissipating heat or securing an additional space.

Although the preferred embodiments of the present invention have been disclosed for illustrative purposes, those skilled in the art will appreciate that various modifications, additions and substitutions are possible, without departing from the scope and spirit of the invention as disclosed in the accompanying claims.

What is claimed is:

1. An air sterilizing and lighting apparatus, comprising:
a main housing configured such that an air blower fan is coupled to a coupling groove formed in a center of a bottom portion of the main housing, sterilization paths are formed at both sides of the coupling groove, and exits are provided at ends of the sterilization paths;
a subsidiary housing configured such that an inlet is formed at a center of the subsidiary housing and through holes for exposure of the sterilization paths of the main housing are formed at both sides of the inlet, and fixedly coupled to the bottom portion of the main housing;
a substrate configured such that an inlet is formed at a center of the substrate, LEDs are fixedly installed on a lower surface of the substrate and UV LEDs are fixedly installed at both sides of an upper surface of the substrate so as to be directed to the sterilization paths of the main housing via the through holes of the subsidiary housing, and fixedly connected to a bottom portion of the subsidiary housing; and
a cover configured such that an inlet is formed at a center of the cover, and fixedly coupled to a bottom portion of the substrate.

2. The air sterilizing and lighting apparatus according to claim 1, wherein titanium oxide plates having vent holes formed therethrough are fixedly coupled to the sterilization paths provided at the sides of the coupling groove of the main housing.

3. The air sterilizing and lighting apparatus according to claim 1, wherein heat-dissipation and guide plates are installed to be located inside the sterilization paths provided at the sides of the coupling groove of the main housing so as to form the sterilization paths in a zigzag form.

4. The air sterilizing and lighting apparatus according to claim 3, wherein the heat-dissipation and guide plates are fixedly installed on the substrate and enter insides of the sterilization paths via the through holes of the subsidiary housing so as to form the sterilization paths in the zigzag form.

5. The air sterilizing and lighting apparatus according to claim 1, wherein a subsidiary cover is fixedly coupled to the inlet of the cover so as to expose the inlet of the cover in a lateral direction.
